(19) United States
(12) Patent Application Publication
(10) Pub. No.: US 2026/0234562 A1
(43) Pub. Date: Aug. 13, 2026

(54) COMPOSITE NANOFIBROUS SCAFFOLD ENHANCES IN VITRO HEPATOCYTES' DRUG CLEARANCE PROFILES TO ALIGN WITH HUMAN PHYSIOLOGY

(71) Applicant: UNIVERSITY OF MARYLAND, BALTIMORE COUNTY, Baltimore, MD (US)

(72) Inventors: Chengpeng CHEN, Baltimore, MD (US); Rudolph PARK, Baltimore, MD (US)

(21) Appl. No.: 19/532,464

(22) Filed: Feb. 6, 2026

Related U.S. Application Data
(60) Provisional application No. 63/755,312, filed on Feb. 7, 2025.

(51) Int. Cl. C12N 5/071; C12N 9/64; D01D 5/00

(52) U.S. Cl. CPC C12N 5/0671; C12N 9/6481; D01D 5/003; C12N 2513/00; C12N 2533/40; C12N 2533/90; C12Y 304/23001; D10B 2331/041

(57) ABSTRACT

An in vitro 3D hepatocyte model that contains a nanofibrous scaffold designed to mimic the extracellular matrix (ECM) of the human liver, both structurally and biochemically, is described. HepaRG hepatocytes cultured on the 3D scaffold exhibited enhanced metabolic activity compared to traditional 2D cultures, indicating improved hepatocyte functionality. This model offers a physiologically relevant platform for hepatocyte studies, underscoring the model's potential to advance preclinical drug development by replicating liver-specific functions in vitro.

### CROSS REFERENCE TO RELATED APPLICATIONS

This application claims priority to U.S. Provisional Patent Application No. 63/755,312 filed on Feb. 7, 2025, in the name of Chengpeng CHEN and Rudolph PARK entitled “Composite Nanofibrous Scaffold Enhances In Vitro Hepatocytes' Drug Clearance Profiles to Align with Human Physiology,” which is hereby incorporated by reference herein in its entirety.

### STATEMENT REGARDING FEDERALLY SPONSORED RESEARCH

This invention was made with United States Government support under Grant Number 1R35GM146779 awarded by the National Institute of General Medical Science. The Government has certain rights in the invention.

### FIELD

The present invention relates to an in vitro 3D hepatocyte model that contains a nanofibrous scaffold designed to mimic the extracellular matrix (ECM) of the an organ such as the human liver, both structurally and biochemically, and methods of making an using same.

### BACKGROUND

Drug development is divided into preclinical and clinical stages. Preclinical studies focus on understanding the mechanisms and targets of candidate drug compounds and validating efficacy and possible toxicity. Drug candidates that pass preclinical screening are routed into clinical trials with human subjects. However, with current technologies, compounds that pass preclinical testing and validation have a low probability of being launched as a drug. This high failure rate, largely caused by the limited predictive power of preclinical models, drives up the cost and time for drug development, but can also lead to unexpected tragic outcomes in clinical trials or even after a drug is marketed.

Currently, preclinical studies rely heavily on cell culture and animal models. Cell culture in well-established containers such as flasks, Petri dishes, and well plates is simple, scalable, relatively cost-efficient, and yields fast results. Nonetheless, growing cells in a 2D monolayer on a flat surface does not provide in vivo cell-cell and cell-extracellular matrix (ECM) interactions, and thus may not represent human physiology. The ECM is a network of micro-/nano-fibers composed of glycoproteins, collagen, fibronectin, elastin, and other macromolecules [Karamanos, 2021; Hussey, 2018]. Emerging evidence suggests that in addition to acting as a nesting niche for cells, the chemical and physical properties of the ECM (e.g., stiffness, microstructures) are key regulators of intracellular biochemical processes [Saraswathibhatla, 2023; Jones, 2021; Huang, 2020; Huang, 2021; Dutta, 2009; Dityatev, 2010]. On the other hand, while lab animals and humans have only slight differences in their genomes, these differences can be significantly amplified during transcriptional and post-transcriptional processes, causing major phenotypic variances [Peaston, 2006], such as in hepatocyte enzyme activity [Ning, 2018; Ning 2019].

Therefore, new preclinical models that align more closely to human in vivo responses are being actively studied. Hitherto, two technologies have mainly been investigated: 3D-cell culture-based physiological models [Gerardo-Nava, 2023] and organs-on-a-chip models [Leung, 2022, Low, 2021]. By incorporating ECM materials such as hydrogels and/or fibrous scaffolds in human cell culture, 3D-cell culture-based physiological models enhance intercellular and cell-ECM interactions, making cell functions and activities more closely match in vivo behaviors [Sackett, 2018; Kolesky, 2016]. For instance, 3D spheroid tumor models exhibit more complex, in vivo-like responses to therapeutics versus flat, 2D cultures [Beller, 2023].

Investigating absorption, distribution, metabolism, elimination, and toxicity (ADMET) is an essential part of drug development [Lin, 2003]. Drug metabolism is mainly carried out by the liver in vivo [Serras, 2021], therefore understanding the metabolism of a drug candidate is important. For example, certain drugs must be metabolized by the liver before they can be excreted, prodrugs need to be metabolized to become active compounds, orally delivered drugs are extensively metabolized in the liver before reaching circulation, and the metabolism rates determine the dosage regimes of a drug.

Accordingly, integrating reliable and predictive liver models in the drug development pipeline will reduce cost burdens and safety issues. Disadvantageously, while toxicity responses are an important component of preclinical testing, hepatocyte models that can reproduce physiological drug metabolism/clearance profiles are limited in literature.

There remains a need in the art for an in vitro 3D hepatocyte model that contains a nanofibrous scaffold designed to mimic the extracellular matrix (ECM) of the human liver, both structurally and biochemically. This model offers a physiologically relevant platform for hepatocyte studies. The model has significant potential to advance preclinical drug development by replicating liver-specific functions in vitro.

### SUMMARY

In some aspects, a method of producing a three-dimensional composite nanofibrous scaffold comprising native extracellular matrix (ECM) components is described, said method comprising:
electrospinning a polymer-containing solution onto a collector to produce a uniform, three-dimensional layer of nanofibers,
   wherein the polymer-containing solution comprises polycaprolactone (PCL) and pepsinated native decellularized extracellular matrix (dECM) powder, and wherein the nanofibers comprise native ECM components, and wherein the nanofibers comprise native ECM components.

In some other aspects, a three-dimensional composite nanofibrous scaffold comprising nanofibers comprising polycaprolactone (PCL) and extracellular matrix (ECM) components of a native tissue is described.

In other aspects, a method of making a three-dimensional tissue comprising a composite nanofibrous scaffold and cells is described, said method comprising:
electrospinning a polymer-containing solution onto a collector to produce a uniform, three-dimensional layer of nanofibers; and
culturing a cell line on and/or within the three-dimensional layer of nanofibers,
   wherein the polymer-containing solution comprises polycaprolactone (PCL) and pepsinated native decellularized extracellular matrix (dECM) powder, and wherein the nanofibers comprise native ECM components, and wherein the cells infiltrate into the three-dimensional layer of nanofibers.

In some other aspects, a three-dimensional tissue is described, said 3D tissue comprising cells and a composite nanofibrous scaffold comprising nanofibers comprising polycaprolactone (PCL) and extracellular matrix (ECM) components of a native tissue, wherein the cells are present on and/or within the composite nanofibrous scaffold.

Other aspects, features and embodiments of the invention will be more fully apparent from the ensuing disclosure and appended claims.

### BRIEF DESCRIPTION OF THE FIGURES

FIG. 1A is an image of a polystyrene sheet with precut holes (14.5 mm diameter) wrapped around a 50 mm wide metal mandrel.

FIG. 1B shows fibers (white) deposited on the exposed metal and polystyrene during electrospinning.

FIG. 1C shows the decellularized extracellular matrix (dECM) inserts cut from the polystyrene sheet.

FIG. 1D is a close-up of a dECM insert, 18.5 mm outer diameter (second laser-cutting after electrospinning), 14.5 mm inner diameter (cut before electrospinning).

FIG. 2A is a SEM image of the composite (PCL+dECM) nanofibrous scaffold.

FIG. 2B illustrates a histogram of the fiber diameter distribution (percentage).

FIG. 2C illustrates picrosirius red staining of PCL (left) and PCL+dECM (right) scaffolds after thorough rinsing. Fluorescence of the scaffolds was scanned by a plate reader (bottom) at 560 nm excitation and 650 nm emission, the scale bar shows fluorescent intensity from 0 (blue) to 37600 (red) units.

FIG. 2D is a FTIR spectra of PCL (blue) and PCL+dECM (orange) fibers. The inset shows the spectra with a zoomed-in view between 1400 and 1700 cm⁻¹.

FIG. 3A illustrates a 3D-printed holder device to house a fiber insert.

FIG. 3B illustrates the CAD designs of the hollow lid.

FIG. 3C illustrates the CAD designs of the scaffold holder, which comprises a base.

FIG. 3D is a picture of a scaffold holder assembly comprising the scaffold holder and the hollow lid.

FIG. 3E is a picture of how the insert holders fit in a standard well-plate for easy integration into cell-culture workflows. The hollow lids were not included to show the inserts and O-rings.

FIG. 4A illustrates cell numbers under the two culture conditions. N>20 for both, error bar=SD. Hepatocytes in a regular well-plate were denoted as “on flat,” and hepatocytes on the 3D PCL+dECM scaffold described herein were denoted as “on dECM.”

FIG. 4B is a confocal microscopy view of hepatocytes on dECM. The cells could infiltrate via the pores between fibers by ~50 μm.

FIG. 4C illustrates quantified albumin production from hepatocytes. N=3 for on flat and 4 for on dECM, error bar=SD.

FIG. 4D illustrates quantified urea production from hepatocytes. N=3 for on flat and 4 for on dECM, error bar=SD.

FIG. 4E illustrates the MTS assay results and analyses. N=4 for both, error bar=SD.

FIG. 5A is a schematic of the major lidocaine pathways.

FIG. 5B illustrates lidocaine clearance by hepatocytes on flat vs. on dECM. N=3 for both, error bar=SD. Hepatocytes in a regular well-plate were denoted as “on flat,” and hepatocytes on our 3D scaffold made of PCL+dECM were denoted as “on dECM.”

FIG. 5C illustrates comparisons of the time needed to remove 50% of lidocaine. N=4 for on flat and 3 for on dECM, N=8 for in vivo.

FIG. 5D illustrates a more detailed comparison of percentage lidocaine being removed at different temporal intervals. N=4 for on flat and 3 for on dECM. Error bar=SD.

FIG. 5E illustrates the comparison of intrinsic clearance rate of lidocaine by hepatocytes on flat and on dECM vs. in vivo data from literature. N=4 for on flat and 3 for on dECM. Error bar=SD.

FIG. 6A is a schematic of the major clozapine pathways.

FIG. 6B illustrates clozapine clearance by hepatocytes on flat vs. on dECM. N=3 for both, error bar=SD. Hepatocytes in a regular well-plate were denoted as “on flat”, and hepatocytes on our 3D scaffold made of PCL+dECM were denoted as “on dECM”.

FIG. 6C illustrates comparisons of the time needed to remove 50% of clozapine. N=4 for on flat and 3 for on dECM, N=9 for in vivo.

FIG. 6D illustrates a more detailed comparison of percentage clozapine being removed at different temporal intervals. N=4 for on flat and 3 for on dECM. Error bar=SD.

FIG. 6E illustrates a comparison of intrinsic clearance rate of clozapine by hepatocytes on flat and on dECM vs. in vivo data from literature. N=4 for on flat and 3 for on dECM. Error bar=SD.

FIG. 7A is a schematic of the major fluoxetine pathways.

FIG. 7B illustrates fluoxetine clearance by hepatocytes on flat vs. on dECM. N=3 for both. Error bar=SD. Hepatocytes in a regular well-plate were denoted as “on flat,” and hepatocytes on our 3D scaffold made of PCL+dECM were denoted as “on dECM.”

FIG. 7C is a schematic showing that the fluoxetine metabolite, S-norfluoxetine, inhibits CYP2D6 in turn, the major enzyme metabolizing fluoxetine.

### DETAILED DESCRIPTION, AND PREFERRED EMBODIMENTS THEREOF

Although the claimed subject matter will be described in terms of certain embodiments, other embodiments, including embodiments that do not provide all of the benefits and features set forth herein, are within the scope of this disclosure as well. Various structural and parameter changes may be made without departing from the scope of this disclosure.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

“About” and “approximately” are used to provide flexibility to a numerical range endpoint by providing that a given value may be “slightly above” or “slightly below” the endpoint without affecting the desired result, for example, +/−5%.

The phrase “in one embodiment” or “in some embodiments” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The term “extracellular matrix” refers to a natural scaffold for cell growth that is prepared by decellularization of tissue found in mammals and multicellular organisms. The extracellular matrix may be a mixture of structural or non-structural biomolecules including, but not limited to, collagens, elastins, laminins, glycosaminoglycans, proteoglycans, antimicrobials, chemoattractants, cytokines and growth factors. In some embodiments, the ECM is derived from a specific organ or biological tissue of interest of at least one of rodent, porcine, feline, canine, bovine, equine, primate, or human. In some embodiments, the ECM is derived from porcine liver tissues, wherein the decellularized liver tissues comprise native extracellular matrix components and thus can provide the physical, mechanical and biochemical environment of a liver for in vitro studies.

As used herein, the specific organ or biological tissue of interest includes, but is not limited to, healthy or unhealthy skin, muscle, vasculature, urethra, bladder, prostate, bone, cartilage, fat, nerve, tumor, cyst, polyp, ribs, stomach, abdominal wall, diaphragm, gallbladder, intestines, bone marrow, liver, eye, vascular bed, aorta, inferior vena cava, superior vena cava, pulmonary arteries, pulmonary veins, portal vein, hepatic veins, hepatic artery, renal arteries, renal veins, femoral arteries, femoral veins, iliac arteries, iliac veins, mesenteric arteries, mesenteric veins, splenic artery, splenic vein, carotid arteries, vertebral arteries, jugular veins, subclavian arteries, subclavian veins, brachiocephalic artery, brachiocephalic vein, coronary arteries, coronary sinus, left atrium, right atrium, left ventricle, right ventricle, mitral isthmus, atrial appendages, papillary muscles, pulmonary trunk, bronchial arteries, segmental and lobar pulmonary vessels, trachea, mainstem bronchi, cerebrospinal fluid spaces, intervertebral disc, nucleus pulposus, annulus fibrosislateral ventricles, third ventricle, fourth ventricle, cerebral aqueduct, central canal of the spinal cord, basilar artery, anterior cerebral artery, middle cerebral artery, posterior cerebral artery, internal carotid arteries, arteries and veins of the Circle of Willis, spinal venous plexus vertebral venous plexus, cancellous bone, liver parenchyma, kidney cortex, kidney medulla, adrenal glands, pancreas, spleen, uterus, vagina, ureter, seminal vesicles, ovaries, testicles, pelvic venous plexuses, and rectum. In some embodiments, the specific organ or biological tissue of interest is the liver.

As used herein, HepaRG® cells is a line of human hepatic progenitors (i.e., undifferentiated human hepatic cells) derived from human hepatocellular carcinoma that retain many characteristics of primary human hepatocytes. HepaRG cells are an immortalized cell line with four main features: they display a full array of functions, responses, and regulatory pathways of primary human hepatocytes including phase I and II, and transporter activities consistent with those found within a population of primary human hepatocytes; they form bile canaliculi; they have the potential to express major properties of stem cells; and they have high plasticity and complete trans differentiation capacity. HepaRG cells may form cellular spheroids that mimic or simulate one or more cellular processes of the liver, which may be stained and/or fluoresced and imaged during in vitro microscopy assays for downstream analysis. Advantageously, HepaRG can be differentiated into human hepatocyte cells in one simple step [Kanebratt, 2008] and numerous pieces of evidence have demonstrated the cells preserve liver-specific genes, protein expression, and functions [Krist, 2018; Kanebratt, 2008; Guillouzo, 2007; Jackson, 2016]. For the purposes of the present disclosure, the phrase “an immortalized human hepatic progenitor that is hepatocellular carcinoma-derived” comprises, consists of, or consists essentially of, the HepaRG cell line.

As used herein, a “differentiated cell,” includes, but is not limited to, a megakaryocyte, an osteoblast, a chondrocyte, an adipocyte, a hepatocyte, a hepatic mesothelial cell, a biliary epithelial cell, a hepatic stellate cell, a sinusoid endothelial cell, a Kupffer cell, a pit cell, a vascular endothelial cell, a pancreatic duct epithelial cell, a pancreatic duct cell, a centroacinous cell, an acinar cell, a islets of Langerhan cell, a cardiac muscle cell, a fibroblast, a keratinocyte, a smooth muscle cell, a type I alveolar epithelial cell, a type II alveolar epithelial cell, a Clara cell, an epithelial cell, a basal cell, a goblet cell, a neuroendocrine cell, a kultschitzky cell, a renal tubular epithelial cell, a urothelial cell, a columnar epithelial cell, a glomerular epithelial cell, an endothelial cell, a podocyte, a mesangium cell, a nerve cell, an astrocyte, a microglia, or a oligodendrocyte.

As used herein, “devoid of” means that none of the indicated substance is intentionally added to or present in the specified product, e.g., the solution. Alternatively, “devoid of” can mean that the amount of the indicated substance in the product is less than about 0.1% (w/w), or less than about 0.05% (w/w), or less than about 0.01% (w/w).

As used herein, “hydrogels” are known in the art as materials hardened through a sol-gel phase transition and are polymers kinetically trapped in a colloidal state. Hydrogels can be natural, semi-synthetic, or synthetic.

As used herein, “stem cells” include embryonic stem cells or induced pluripotent stem cells.

As used herein, “on and/or within” the three-dimensional layer of nanofibers or the composite nanofibrous scaffold is intended to correspond to cells that remain on the surface of the 3D layer of nanofibers, cells that are within (or have infiltrated) the 3D layer of nanofibers, or both. In some embodiments, the cells infiltrate to a depth of about 50 m in the 3D layer of nanofibers.

Broadly, a 3D model based on a composite nanofibrous scaffold that mimics both the microstructures of the native organ ECM and its biochemical composition are described herein.

Scaffolds provide a structural framework that mimics the ECM found in biological tissues and allow cells to adhere, proliferate, and differentiate on and/or within a 3D environment, mimicking and/or simulating the processes and/or functions of an organ of a subject or patient.

Advantageously, the composite nanofibrous scaffolds can be used for in vitro testing of the efficacy, non-toxicity and/or toxicity of various compounds in relation to an organ instead of in vivo testing. In some embodiments, the composite nanofibrous scaffolds may include immortalised cell lines that have been developed to mimic or simulate a particular organ of a subject.

In some embodiments, the composite nanofibrous scaffold is produced using electrospinning. Electrospinning may be used to transform many types of polymers into electrospun nanofiber networks which mimic the ECM with respect to fiber diameters, porosity, and mechanical properties.

Accordingly, in a first aspect, a method of producing a three-dimensional composite nanofibrous scaffold comprising native extracellular matrix (ECM) components is described, said method comprising:
electrospinning a polymer-containing solution onto a collector to produce a uniform, three-dimensional layer of nanofibers,
   wherein the polymer-containing solution comprises polycaprolactone (PCL) and pepsinated native decellularized extracellular matrix (dECM) powder, and wherein the nanofibers comprise native ECM components.

In some embodiments, the polymer-containing solution is prepared by combining (a) a first solution comprising polycaprolactone (PCL) and (b) a second solution comprising pepsinated native dECM powder. In some embodiments, the first solution further comprises 2,2,2-trifluoroethanol (TFE) or hexafluoropropanol, preferably TFE. In some embodiments, the second solution further comprises TFE or hexafluoropropanol and at least one weak acid, for example, acetic acid, formic acid, succinic acid, lactic acid, citric acid, or oxalic acid. In some embodiments, the second solution further comprises TFE and acetic acid. Regardless of how the polymer-containing solution is prepared, the ratio of PCL to dECM in the polymer-containing solution is about 1-3:1 (w/v), or about 3:1 (w/v), or about 2:1 (w/v), or about 1.5:1 (w/v), or about 1:1 (w/v). In some embodiments, the polymer-containing solution comprises about 5-15% PCL (w/v), or about 6-14% PCL (w/v), or about 7-13% PCL (w/v), or about 8-12% PCL (w/v), or about 9-11% PCL (w/v), or about 10% PCL (w/v). In some embodiments, the polymer-containing solution comprises about 5-15% dECM (w/v), or about 6-14% dECM (w/v), or about 7-13% dECM (w/v), or about 8-12% dECM (w/v), or about 9-11% dECM (w/v), or about 10% dECM (w/v).

In some embodiments, the dECM is derived from native organs or biological tissues of interest. In some embodiments, the dECM is derived from native liver tissues, for example, porcine, bovine, or human liver tissue. In some embodiments, the dECM is derived from native liver tissues, for example, porcine liver tissue. In some embodiments, the method of preparing the pepsinated native dECM powder comprises: decellularizing the native tissues to obtain dECM; lyophilizing and grinding the dECM to obtain powdered dECM; digesting the powdered dECM with digestion solution to obtain a digested dECM; and lyophilizing and grinding the digested dECM to obtain the pepsinated native dECM powder. In some embodiments, the method of preparing the pepsinated native dECM powder comprises: decellularizing the native tissues to obtain dECM; lyophilizing and grinding the dECM to obtain powdered dECM; digesting the powdered dECM with digestion solution to obtain a digested dECM; denaturing the pepsin; and lyophilizing and grinding the digested dECM supernatant to obtain the pepsinated native dECM powder. In some embodiments, the digestion solution comprises pepsin. In some embodiments, the digestion solution comprises pepsin and at least one acid, e.g., dilute HCl, dilute sulfuric acid, dilute nitric acid, or any other acid that will reduce the pH to about 2. In some embodiments, the pepsin is denatured using a strong base such as a hydroxide, e.g., NaOH, to produce a weakly basic solution having a pH in a range of about 7.5-8.5. Other methods of obtaining decellularized ECM, and hence pepsinated native dECM powder are known in the art and the method described herein is not intended to limit same.

In some embodiments, the ECM of the composite nanofibrous scaffold mimics the organ or biological tissue of interest in terms of microstructures and biochemical composition. In some embodiments, the composite nanofibrous scaffold is biologically relevant to the liver ECM in terms of microstructures and biochemical composition. In some embodiments, the diameter of the nanofibers is in a range of about 100-500 nm. In some embodiments, the composite nanofibrous scaffold is devoid of hydrogel or a hydrogel-based material. In some embodiments, the composite nanofibrous scaffold is devoid of alginate. In some embodiments, the composite nanofibrous scaffold is devoid of polyvinyl alcohol (PVA) nanofibers. In some embodiments, the composite nanofibrous scaffold is devoid of MATRIGEL® (from Corning Inc. of Corning, NY) and/or GELTREX® (from Thermo Fisher Scientific Inc. of Waltham, MA)). In some embodiments, the composite nanofibrous scaffold is devoid of gelatin or a gelatin-based material (e.g., gelatin methacryloyl (GelMA)). In some embodiments, the composite nanofibrous scaffold is devoid of a polyethylene glycol (PEG)-based material (e.g., PEGDA). In some embodiments, the composite nanofibrous scaffold is devoid of more than one of hydrogel or a hydrogel-based material, alginate, polyvinyl alcohol (PVA) nanofibers, MATRIGEL® and/or GELTREX®, gelatin or a gelatin-based material, a polyethylene glycol (PEG)-based material, all of the above, or any combination thereof.

Electrospinning is well known in the art. Broadly, the instrumentation necessary for electrospinning is well known in the art include a high voltage power supply, a spinneret, and a collector. The spinneret may be a capillary tube with a pipette or needle having a small diameter. The collector may be a metal collecting plate. One electrode of the high voltage power supply is placed into a polymer solution and the other electrode is attached to the collector. An electric field is applied to the end of the capillary tube that contains the polymer-containing solution held by its surface tension and forms a charge on the surface of the liquid. As the intensity of the electric field increases, the hemispherical surface of the fluid at the tip of the capillary tube elongates, forming a conical shape known as a Taylor cone. A critical value is attained upon a further increase in the electric field, where the repulsive electrostatic force overcomes the surface tension and the charged jet of fluid is ejected from the tip of the Taylor cone. The discharged polymer solution jet is unstable and elongates as a result, allowing the jet to become very long and thin. Charged polymer fibers solidify with solvent evaporation. Randomly oriented nanofibers are collected on the collector. Nanofibers can also be collected in a highly aligned fashion using specialized collectors such as a rotating drum, mandrel, metal frame, or a two-parallel plate system. Parameters such as jet stream movement and polymer concentration must be controlled to produce nanofibers with uniform diameters and morphologies.

In some embodiments, a three-dimensional composite nanofibrous scaffold prepared according to the method of the first aspect is disclosed. In some embodiments, the three-dimensional composite nanofibrous scaffold comprises nanofibers comprising polycaprolactone (PCL) and extracellular matrix (ECM) components of a native tissue. In some embodiments, the ECM is derived from native organs or biological tissues of interest. In some embodiments, the ECM is derived from native liver tissues, for example, porcine liver tissue. In some embodiments, the diameter of the nanofibers is in a range of about 100-500 nm. In some embodiments, the electrospun composite nanofibrous scaffold has a non-woven structure, wherein the fibers have a high surface area, the scaffold has a high porosity In some embodiments, the composite nanofibrous scaffold is devoid of hydrogel or a hydrogel-based material. In some embodiments, the composite nanofibrous scaffold is devoid of alginate. In some embodiments, the composite nanofibrous scaffold is devoid of polyvinyl alcohol (PVA) nanofibers. In some embodiments, the composite nanofibrous scaffold is devoid of MATRIGEL® (from Corning Inc. of Corning, NY) and/or GELTREX® (from Thermo Fisher Scientific Inc. of Waltham, MA)). In some embodiments, the composite nanofibrous scaffold is devoid of gelatin or a gelatin-based material (e.g., gelatin methacryloyl (GelMA)). In some embodiments, the composite nanofibrous scaffold is devoid of a polyethylene glycol (PEG)-based material (e.g., PEGDA). In some embodiments, the composite nanofibrous scaffold is devoid of more than one of hydrogel or a hydrogel-based material, alginate, polyvinyl alcohol (PVA) nanofibers, MATRIGEL® and/or GELTREX®, gelatin or a gelatin-based material, a polyethylene glycol (PEG)-based material, all of the above, or any combination thereof.

In some embodiments of the first aspect, the composite nanofibrous scaffold is three-dimensional. In some embodiments, the composite nanofibrous scaffold has an approximately cylindrical shape defined by a height and a two-dimensional base, wherein the two-dimensional base is selected from the group consisting of circular, square, triangular, elliptical, polygonal, or any other shape depending on what is required by the user, and the height is about 20 to about 500 m. In some embodiments, the composite nanofibrous scaffold is a circular cylinder and is sized to fit within a composite nanofibrous scaffold holder assembly as described herein, or the equivalent thereof, wherein the composite nanofibrous scaffold holder assembly standard fits into a well-plate for easy integration into cell-culture workflows. For example, the composite nanofibrous scaffold is obtained by electrospinning the polymer-containing solution onto a mandrel that is covered by a polystyrene sheet having holes of the preferred 2D shape having a first size, e.g., a first diameter, wherein the holes define the nanofiber deposition locations. In some embodiments, the composite nanofibrous scaffold deposited can be removed from the polystyrene sheet and used as is. In some embodiments, the composite nanofibrous scaffold further comprises a polystyrene (PS) ring having a layer of nanofibers thereon, i.e., on top, which is obtained by placing the polystyrene sheet comprising the deposited composite nanofibrous scaffolds in a laser cutter to cut out a second size, e.g., a second diameter, comprising the deposited composite nanofibrous scaffolds inserts, wherein the second size is greater than the first size, e.g., the second diameter is greater than the first diameter. Because the second size is greater than the first size, the laser-cutting process heat-fuses the edges of the nanofiber and polystyrene layers, producing a semi-rigid or rigid ring around the composite nanofibrous scaffolds for easier handling. It should be appreciated by the person skilled in the art that this embodiment is not intended to limit the composite nanofibrous scaffold or uses thereof.

In some embodiments, a 3D hepatocyte model based on a composite nanofibrous scaffold that mimics both the microstructures of the native porcine liver ECM and its biochemical composition is described herein. In some embodiments, instead of using primary human hepatocytes, which have limited availability and are difficult to trace (e.g., donor health/medical history), or hepatocyte-like cells derived from pluripotent stem cells, the model described herein uses HepaRG hepatocytes [Krist, 2018; Stanley, 2022].

Accordingly, in a second aspect, a method of making a three-dimensional tissue comprising a composite nanofibrous scaffold and cells is described, said method comprising:
electrospinning a polymer-containing solution onto a collector to produce a uniform, three-dimensional layer of nanofibers; and
culturing a cell line on and/or within the three-dimensional layer of nanofibers,
   wherein the polymer-containing solution comprises polycaprolactone (PCL) and pepsinated native decellularized extracellular matrix (dECM) powder, and wherein the nanofibers comprise native ECM components, and wherein the cells infiltrate into the three-dimensional layer of nanofibers.

In some embodiments, the method of electrospinning a polymer-containing solution onto a collector to produce a uniform, three-dimensional layer of nanofibers, wherein the polymer-containing solution comprises polycaprolactone (PCL) and pepsinated native decellularized extracellular matrix (dECM) powder, and wherein the nanofibers comprise native ECM components, is as described hereinabove for the first aspect. In some aspects, the three-dimensional layer of nanofibers, wherein the polymer-containing solution comprises polycaprolactone (PCL) and pepsinated native decellularized extracellular matrix (dECM) powder, and wherein the nanofibers comprise native ECM components, is obtained using other methods in the art.

In some embodiments, the cell line that is cultured is an immortalized human hepatic progenitor that is hepatocellular carcinoma-derived, i.e., HepaRG. In some embodiments, the cell line that is cultured is HUH7 human hepatocarcinoma cells. In some embodiments, the cell line that is cultured is HepG2 cells. In some embodiments, the cell line that is cultured is primary human hepatocytes. In some embodiments, the cell line that is cultured is primary cynomolgus hepatocytes. In some other embodiments the lcell line is selected from the group consisting of the Huh6, Huh7.5.1, Hep3B.1-7, SkHep1, C3A, PLC/PRF/5 and SNU-398 cell lines or optionally a combination with another cell line such as LX2 cells, THP1 cells or another cell line or primary cells such as human Kupffer cells or human myofibroblasts or liver sinusoidal endothelial cells. It is to be understood by the person skilled in the art that any type of cells can be cultured in or on the composite nanofibrous scaffold described herein. In some embodiments, the cells that infiltrate into the three-dimensional layer dimensional layer of nanofibers. In some embodiments, the culturing is effectuated using the composite nanofibrous scaffold holder assembly, inserted into a standard well-plate, as described hereinafter. In other embodiments, the culturing is effectuated using other methods known in the art. Methods of cell differentiation are well known in the art.

Accordingly, in some embodiments of the second aspect, a three-dimensional tissue comprising a composite nanofibrous scaffold and differentiated cells prepared according to the method of the second aspect is described. In some embodiments, the differentiated cells includes hepatocytes. In some embodiments, the three-dimensional tissue comprises cells and a composite nanofibrous scaffold comprising nanofibers comprising polycaprolactone (PCL) and extracellular matrix (ECM) components of a native tissue, wherein the cells are present on and/or within the composite nanofibrous scaffold.

In some embodiments, the differentiated cells do not include primary human hepatocytes and hepatocyte-like cells derived from embryonic or pluripotent stem cells.

As will be discussed in the Examples, HepaRG progenitor cells were successfully seeded and differentiated on the scaffold and exhibited typical hepatic functions. By treating the differentiated hepatocytes with well-documented drugs that need to be metabolized before excretion, it was discovered that hepatocytes on the model described herein reproduced in vivo drug clearance rates, while cells in conventional well plates (2D culture) showed significantly slower drug clearance and reduced clearing capacities. Further modular 3D-printed holders and batch-produced scaffold inserts are described herein which can be assembled in seconds and housed in standard well plates. The ease of use of the fibrous scaffold and its ability to modulate in vivo-like functions of HepaRG hepatocytes represents a novel and significant improvement in preclinical modeling.

Accordingly, in a third aspect, a method of determining the extent of drug metabolism or clearance using the three-dimensional tissue of the second aspect as an in vitro model is described herein. In some embodiments, the method of the third aspect is effectuated using the composite nanofibrous scaffold holder assembly described hereinafter.

In some other embodiments, the composite nanofibrous scaffold described herein is useful in disease modeling research that implements various intractable liver diseases (hepatocirrhosis, liver fibrosis, non-alcoholic hepatitis, etc.) in vitro and reveals the mechanisms thereof, and establishment of a transplantation treatment platform.

In a fourth aspect, a composite nanofibrous scaffold holder assembly is described herein, said assembly comprising:
a scaffold holder being a walled container having a first wall height, a first open end, optionally a first closed end, a first inside wall, and a first outside wall, wherein the first inside wall comprises a first connecting means;
a hollow lid comprising a second wall height, a second inside wall, a second outside wall, and two open ends, wherein the hollow lid has a shape that corresponds to that of the scaffold holder and the second outside wall comprises a second connecting means which is complementary to the first connecting means such that the hollow lid can be inserted into the scaffold holder and connected thereto.

In some embodiments, the scaffold holder assembly has a circular cylindrical shape. In some embodiments, the scaffold holder assembly has a circular cylindrical shape such that it fits in a standard well-plate for easy integration into cell-culture workflows. It should be appreciated however that the scaffold holder assembly can have a square cylindrical shape, a oval cylindrical shape, a polygonal cylindrical shape, or any other shape as understood by the person skilled in the art. In some embodiments, the scaffold holder has a first closed end, i.e., a base. In some other embodiments, the scaffold holder has two open ends, depending on how the skilled artisan intends to use the scaffold holder assembly. In some embodiments, the first and second connecting means comprises complimentary continuous threads or other locking mechanisms. In some embodiments, prior to connecting the hollow lid to the scaffold holder, a composite nanofibrous scaffold, as described herein in the first aspect, is inserted into the scaffold holder, followed by an O-ring, e.g., a silicone O-ring, and then the hollow lid is attached to the scaffold holder to create a tight seal. It should be appreciated that the composite nanofibrous scaffold and the O-ring both have shapes that are complimentary to the scaffold holder (and the hollow lid). In some embodiments, the scaffold holder further comprises a step 10 that is positioned within the scaffold holder at the first closed end and is abut to the first inside wall, creating a step upon which the composite nanofibrous scaffold can rest. In some embodiments, the step is removable, for example a second O-ring. In some other embodiments, the scaffold holder is a monolithic piece comprising the step 10. It should be appreciated that the hollow lid, the scaffold holder, and the O-ring are made from biocompatible materials, as well understood by the person skilled in the art. Further, the hollow lid, the scaffold holder, and the O-ring should be sterilizable, as well understood by the person skilled in the art.

### EXAMPLES

### Materials and Methods

Decellularized ECM Extraction. Fresh porcine liver tissues were obtained from local slaughterhouses, cut into ~1 cm³ cubes, and put in a 2 L Erlenmeyer flask. The cubes were rinsed with doubly deionized (DDI) water then soaked in 1.5 L decellularization solution composed of 0.5% Triton X-100 (A16046.AP, Thermo Fisher, Pittsburgh, PA, USA) and 47.6 mM ammonium hydroxide (AX1303-3, MilliporeSigma, St. Louis, MO, USA) and placed on a stir plate in 4° C., stirring at 180 rpm. The decellularization solution was changed every 24 hours for 7 days. The dECM was then thoroughly rinsed with DDI water, patted dry with paper towels, placed in two 250 mL weigh boats (10803-170, VWR International, Radnor, PA, USA), and frozen at −80° C., after which it was lyophilized and ground into a coarse powder. A total of 300-400 mg of coarsely powdered dECM was digested with 40 mL of 3500 U/mL pepsin (P7000, MilliporeSigma, St. Louis, MO, USA) in 0.01 M HCl solution for 24 hours at 37° C. on an orbital shaker, 60 rpm. The solution was neutralized with 0.1 M NaOH to pH 8.0 to permanently denature the pepsin. The digested dECM solution was centrifuged for 20 min at 10,000×g, 4° C. in a high-speed centrifuge (Avanti J-E, Beckman Coulter, Brea, CA, USA), the supernatant collected in 50 mL conical tubes, lyophilized for 72 hours to obtain pepsinated dECM powder, which was stored in −20° C.

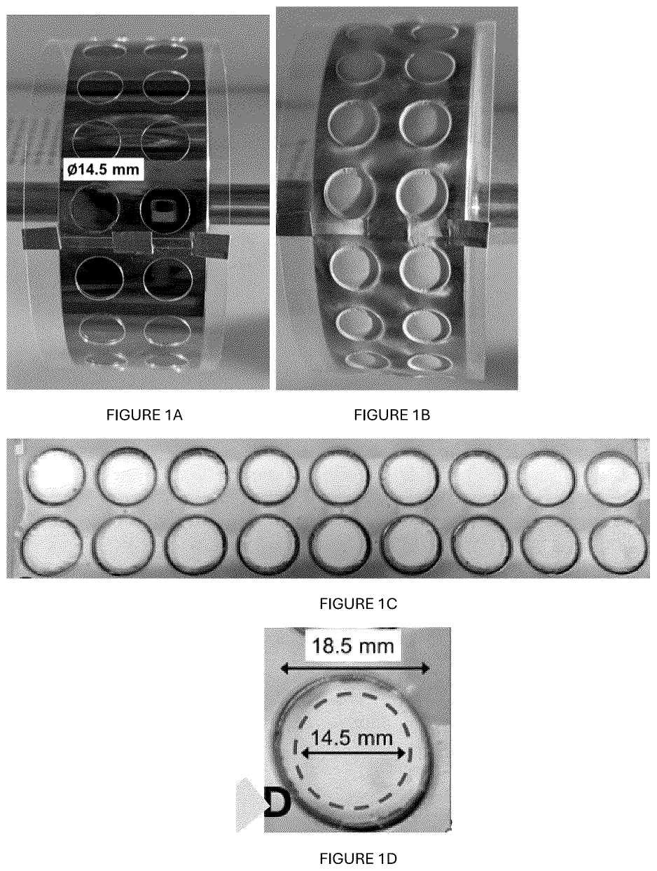

dECM Insert Preparation. The dECM electrospinning solution was made by combining two solutions: Solution 1 comprised 20% w/v polycaprolactone (PCL) with an average molecular weight 80,000 (440744, MilliporeSigma, St. Louis, MO, USA) in 2,2,2-trifluoroethanol (TFE) (A10788, Thermo Fisher, Pittsburgh, PA, USA); Solution 2 comprised 20% w/v pepsinated dECM powder in TFE+5% acetic acid (AC42322, Fisher Scientific, Pittsburgh, PA, USA). 1.5 mL of Solution 2 was pipetted into 1.5 mL of Solution 1 to produce 3.0 mL of the dECM electrospinning solution (10% w/v PCL in TFE, 10% w/v dECM in TFE+2.5% acetic acid). Electrospinning was performed on the TL-Pro-BM electrospinning machine (Tong Li, Shenzhen, China). As shown in FIG. 1A, a 50 mm wide metal mandrel was covered by a polystyrene (PS) sheet with 14.5 mm diameter circular holes cut with a Hobby 5S laser cutter (Full Spectrum Laser, Las Vegas, NV, USA). The circular holes defined the nanofiber deposition locations. A 5 mL Leur-Lok syringe (30964, BD, MA, USA) was used to pump the electrospinning solution through a 24 G metal blunt needle placed 17 cm from the surface of the mandrel. The syringe pump was set to a flow rate of 0.25 mL/hr. The mandrel was rotated at 300 rpm, and the voltage of the mandrel was set to −5 kV, while the needle was set to +17 kV. The electrospinning was conducted for 4 hours. The needle scanned back and forth across the width of the mandrel at a rate of 5 mm/s to deposit a uniform layer of nanofibers (FIG. 1). After the electrospinning was completed, the PS sheet was placed in the laser cutter to cut out circular inserts of 18.5 mm diameter (FIGS. 1C-1D). The laser-cutting process also heat-fused the edges of the nanofiber and PS layers.

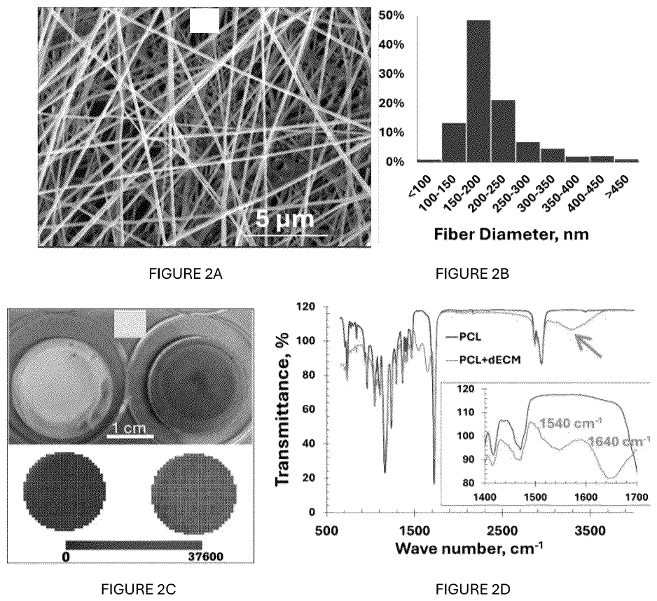

Scanning Electron Microscope (SEM) Imaging of the Scaffolds. An SEM (FEI Nova NanoSEM 450, Thermo Fisher, Pittsburgh, PA, USA) was used to image the dECM inserts at high (2,000×-20,000×) magnification. An insert was mounted on a pin stub SEM mount (75210, Electron Microscopy Sciences, Hatfield, PA, USA), sputter coated with gold, and imaged on the SEM. The DiameterJ plugin for ImageJ/FIJI was used to measure the diameters of the fibers in SEM images (FIGS. 2A and 2B).

Picrosirius Red (PSR) Staining. The Picrosirius Red Stain Kit (24901, Polysciences, Warrington, PA, USA) was used to stain collagen in the scaffold inserts. The insert was rinsed with DDI water then soaked in 0.5 mL of Solution B (Picrosirius Red F3BA stain) for 60 minutes. The insert was then rinsed with Solution C (0.1 N HCl) for 1 minute, twice, followed by rinsing in DDI water and then in 70% ethanol for 60 s. The stained inserts were then imaged or placed in a plate reader (SpectraMax i3x, Molecular Devices, San Jose, CA, USA) with the well-scanning mode to detect PSR fluorescence signals (560 nm excitation, 650 nm emission, FIG. 2C).

Collagen I Antibody Labelling. The CoraLite Plus 488-conjugated Collage Type I Monoclonal antibody (CL488-67288, Proteintech, Rosemont, IL, USA) was used to label the dECM inserts to visualize collagen from the extracted ECM in the nanofibers. The inserts were rinsed 3× with PBS, fixed for 10 min in 4% paraformaldehyde solution, rinsed 3× in wash buffer (PBS+0.1% Tween 20) for 5 min, and gently rocked in blocking solution (PBS+0.1% Tween 20+1% bovine serum albumin (0332-25G, VWR International, Radnor, PA, USA)) for 60 min at room temperature. The blocking solution was then replaced with 1 mL of antibody solution (1:200 dilution of antibody in blocking solution). The insert was rocked gently overnight at 4° C., covered with foil to protect it from light. The insert was then washed 3× in wash buffer for 5 min and placed on a WillCo glass bottom dish (HBSB-5040, WillCo Wells, Amsterdam, The Netherlands) with 20 μL SlowFade Diamond Antifade Mountant (S36972, Thermo Fisher, Pittsburgh, PA, USA).

Confocal Microscopy. A confocal laser scanning microscope (LSM 900, Zeiss, Oberkochen, Germany) was used to image the fluorescence from PSR-stained and collagen I antibody-labeled inserts. A 561 nm laser was used to excite the PSR-stained sample with a detection band of 613-700 nm. A 488 nm laser was used to excite the CoraLite 488 fluorophore conjugated to the Collagen I antibody with a detection band of 505-550 nm. A 63× objective (oil immersion) was used for both the PSR and CoraLite 488 imaging.

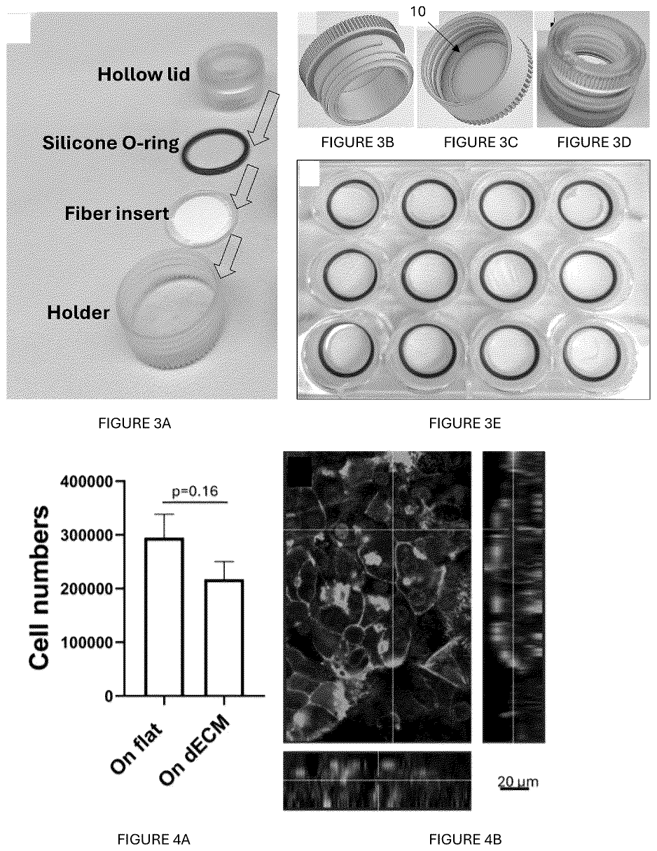

3D-printed Insert Holder Assembly. The dECM insert holder assembly was composed of an insert holder (FIGS. 3A, 3C) and a hollow lid (FIGS. 3A, 3B) that were screwed together, with a silicone O-ring (19 mm OD, 16 mm ID) (FIGS. 3A, 3D, 3E) to create a tight seal. The insert holder and hollow lid were 3D printed (Form 3B, Formlabs, Somerville, MA, USA) using the biocompatible BioMed Clear Resin (RS-C2-BMCL-01, Formlabs, Somerville, MA, USA). Prior to seeding cells, the 3D-printed parts and O-rings were sterilized by autoclaving. The scaffold inserts were sterilized inside a biological safety cabinet (BSC) by soaking in 70% ethanol and placed in the insert holder to dry under UV light. After drying, an O-ring was placed on the insert and compressed by screwing the hollow lid into the insert holder. The assembly was then placed in a 12-well plate (10861556, VWR International, Radnor, PA, USA) (FIG. 3E, lid removed for clarity).

HepaRG Culture and Differentiation. Undifferentiated HepaRG cells (HPR101, Biopredic International, Saint-Gregoire, France) were thawed and maintained in the growth medium composed of HepaRG growth medium supplement with antibiotics (ADD710C, Biopredic International) in Williams E medium with GlutaMAX supplement (32551020, Thermo Fisher, Pittsburgh, PA, USA). The growth medium was refreshed every 2-3 days over 7 days of proliferation in a T75 flask. The flask was then rinsed twice with 5 mL PBS prior to adding 5 mL of 37° C. trypsin (25200056, Thermo Fisher, Pittsburgh, PA, USA) and incubated at 37° C. for 10 min. Growth media (5 mL) was added to the flask to neutralize the trypsin, and the contents of the flask were transferred to a 15 mL conical tube and centrifuged for 5 min @2,000 rpm (SCL456, Southwest Science, Trenton, NJ, USA). The supernatant was decanted, and the cell pellet was resuspended in 5-7 mL of growth medium by gentle pipetting. The cells were counted using the EVE automated cell counter (NanoEntek, Seoul, South Korea), and the cell suspension was diluted to 500,000 cells/mL. For the flat culture condition, the cell suspension was pipetted into four wells of a 12-well plate (1 mL into each well). For the 3D insert culture condition, the cell suspension was pipetted into four dECM insert holder assemblies in the same 12-well plate (1 mL into each assembly).

The growth medium for the flat and dECM inserts was refreshed 6-18 hours after seeding, and then every 2-3 days over 7 days of proliferation. To start differentiation, the growth medium was replaced with 1 mL of 50% growth medium and 50% differentiation medium composed of HepaRG differentiation medium supplement with antibiotics (ADD711C, Biopredic International, Saint-Gregoire, France) in Williams E medium with GlutaMAX supplement (32551020, Thermo Fisher, Pittsburgh, PA, USA). Afterward, the medium was replaced with 1 mL differentiation medium every 2-3 days.

Urea Measurement. Urea in phenol red-free cell differentiation medium was quantified using a colorimetric Urea Assay Kit III (MAK471, MilliporeSigma, St. Louis, MO, USA). Phenol red-free differentiation medium was composed of HepaRG differentiation medium supplement with antibiotics (ADD711C, Biopredic International) in phenol red-free Williams E medium (A1217601, Thermo Fisher, Pittsburgh, PA, USA) and GlutaMAX 100× supplement (35050061, Thermo Fisher, Pittsburgh, PA, USA) diluted to 1× final concentration. An aliquot of 0.75 mL of 24-hr culture medium was collected for urea quantification following the assay kit's manual.

Albumin Assay. The Human Albumin ELISA Kit (ab179887, Abcam, Cambridge, UK) was used to quantify albumin in the cell culture media according to vendor protocols. A 1:400 dilution of the 24-hour culture sample was used for the ELISA assay.

MTS Assay. The MTS Assay Kit (ab197010, Abcam, Cambridge, UK) was used to measure the metabolic activity of differentiated HepaRG cells 13-43 days after differentiation. For each well of flat or dECM insert culture, the differentiation medium was replaced with 500 μL of phenol red-free differentiation medium+50 μL MTS Reagent Solution. A blank with 20 μL MTS Reagent Solution added to 200 μL of phenol red-free differentiation medium was also prepared. The cell culture and blank were incubated at 37° C. for 2 hours, rocking at 20 rpm, 10° tilt (02217765, Fisher Scientific, Pittsburgh, PA, USA). 220 μL of the cell culture and blank were pipetted into a 96-well plate, and the absorbance was measured on a plate reader (SpectraMax i3x, Molecular Devices, San Jose, CA, USA) at 490 nm.

Drug Metabolism Analysis. A 1 μM solution of lidocaine (L7757, MilliporeSigma, St. Louis, MO, USA) was prepared using a stock solution of 20 mM lidocaine in DMSO (042780.AK, Thermo Scientific, Pittsburgh, PA, USA) that was filter sterilized. The flat and dECM inserts with the HepaRG cells were cultured in 0.75 mL of 1 M lidocaine in the differentiation medium for 24 hours with 50 L samples of media taken at 0, 2, 4, 8, and 24 hours. The 12-well plate containing the flat and dECM insert cell cultures was rotated on an orbital shaker (KJ-201BD, Kang Jian Medical, Taizhou City, China) at 30 rpm in a humid 37° C. incubator, 5% CO₂. A 400 nM solution of clozapine (C2460000, MilliporeSigma, St. Louis, MO, USA) was prepared using a stock solution of 30.6 mM clozapine in DMSO (042780.AK, Thermo Scientific, Pittsburgh, PA, USA) that was filter sterilized. The flat and dECM inserts with the HepaRG cells were cultured in 1 mL of 400 nM clozapine in the differentiation medium for 24 hours with 50 L samples of media taken at 0, 2, 4, 8, and 24 hours. The 12-well plate containing the flat and dECM insert cell cultures was rocked at 20 rpm, 10° tilt (02217765, Fisher Scientific, Hampton, NH, USA) in a humid 37° C. incubator, 5% CO₂. A 400 nM solution of fluoxetine (F07501G, Fisher Scientific, Hampton, NH, USA) was prepared using a stock solution of 10 mM fluoxetine in DMSO (042780.AK, Thermo Scientific, Pittsburgh, PA, USA) that was filter sterilized. The flat and dECM inserts with the HepaRG cells were cultured in 1 mL of 400 nM of fluoxetine in the differentiation medium for 72 hours with 50 μL of supernatant taken at 0, 4, 8, 24, 48, and 72 hours. The 12-well plate containing the flat and dECM insert cell cultures was rocked at 20 rpm, 10° tilt on a rocking shaker (02217765, Fisher Scientific, Hampton, NH, USA) in a humid 37° C. incubator, 5% CO₂.

Standards of each drug were prepared, and 50 L of each standard was collected at the same time as the 0-hour sample. Each 50 L sample and standard was pipetted into a 1.5 mL microtube and diluted with 0.45 mL LC/MS Grade Methanol (A456-1, Fischer Scientific, Hampton, NH, USA). The samples were stored at −80° C. for 2 hr to precipitate proteins, the microtubes were then centrifuged (5415 D, Eppendorf, Hamburg, Germany) for 15 min @13,200 rpm in 4° C. and 0.35 mL of the supernatant was pipetted into a 2 mL screw thread Autosampler vial (033919, Fisher Scientific, Hampton, NH, USA) and capped with an autosampler vial cap (13622182, Fisher Scientific, Hampton, NH, USA). The vials were loaded onto the autosampler rack of a triple quadrupole LC-MS/MS (LCMS-8045, Shimadzu Corporation, Kyoto, Japan) for drug analysis. Using multiple reaction monitoring (MRM) in positive mode, the following precursor and product m/z ratios were used based on analyzing the standards: lidocaine (235.1 and 86.1), clozapine (327.1 and 269.75) and fluoxetine (310.1 and 43.95). The sample injection volume was 3 μL for all three drugs, and a Luna Omega 1.6 μm Polar C18 column with an inner diameter of 50×2.1 mm (OOB-47480AN, Phenomenex, Torrance, CA, USA) was used for all three drugs. Mobile phase A was LC/MS grade water with 0.6% formic acid, and mobile phase B was LC/MS grade methanol with 0.6% formic acid. For each drug, a standard curve was calculated and used to quantify the concentration of the media samples taken at various time points.

In vivo data estimation from the literature. In vivo clearance curves of lidocaine and clozapine were taken from following literature of in vivo studies. Figures for drug concentration in blood (serum or plasma) versus time were reanalyzed to obtain the human in vivo data. For intrinsic clearance data, additional literature was analyzed. For reports with the unit per kg, the numbers were multiplied by 70 kg, the average weight of a healthy adult. To normalize the in vivo intrinsic clearance data in the literature to per cell, the published results that 1 g human liver tissue contains 116×10⁶ hepatocytes [Wilson, 2003] and an adult human liver weighs 1561 g on average [Molina, 2012] were used.

Cell Counting. Cells cultured on flat surfaces and on dECM inserts were rinsed in PBS, lysed in 0.3 mL RIPA buffer (J52624, Thermo Fisher, Pittsburgh, PA, USA) diluted to 1×, and collected into microtubes followed by 90 sec of sonication (2 sec on, 4 sec off) using an ultrasonic homogenizer (Pulse 150, Benchmark Scientific, South Plainfield, NJ, USA). The lysed cell solution was then centrifuged for 12 min at 13,200 rpm at 4° C. (5415 D, Eppendorf, Hamburgh, Germany). An aliquot of 10 μL of the supernatant was used in a Bradford assay (23246, Thermo Fisher, Pittsburgh, PA, USA) to quantify the cell number (OD=595 nm). A calibration curve between known cell numbers (EVE, NanoEntek, Seoul, South Korea) and the Bradford assay signals was prepared to elucidate cell numbers in the lysed samples.

$${C = {C}_{0} {e}^{- kt}}_{C is apparentconcentrationof a drug , {C}_{0} is the initial concentration , t is time in hr} \qquad Equation 1$$

$${CL = k V}_{CL is clearance , k is constantfrom Equation 1 , V is distribution volume} \qquad Equation 2$$

Calculations and Statistics. Intrinsic clearance rates (CL) were calculated from experimental data (remaining concentrations as a function of time) with the following equations [Hedaya, 2023]. The volume of media in the experiments, which was 750 μL for lidocaine and 1000 μL for clozapine, was used for V. The CL was then normalized to per cell based on the cell counting protocol above.

For statistical analyses, the replicate numbers and error bar information were detailed in figure captions. Most error bars were standard deviation (SD). All statistical analysis and figures were generated using GraphPad Prism (GraphPad Software, Boston, MA, USA). Student t-tests and ANOVA were applied for significance analyses. The p values were noted between data groups in the figures wherever necessary. A significant difference was determined only when a p-value was less than 0.05.

### Results and Discussion

The scaffold and setup. The purpose of this example was to create a scaffold biologically relevant to the liver ECM in terms of microstructures and biochemical composition. Literature suggests that the native liver ECM is a fibrous network, with estimated ECM fiber diameters ranging from 100 to 500 nm [Baiocchini, 2016; Mazza, 2015; Gupta, 2024]. Electrospinning was used to generate nanofibers similar in thickness to those in the native ECM. Electrospinning transforms a polymer solution into nano-/micro-fibers by pumping the solution through a syringe needle at a high voltage, forming a Taylor cone [Xue, 2019]. By varying parameters such as voltage, needle diameter, flow rate, and distance to the collecting platform, the fiber size, orientation, porosity, and layer thickness can be tuned [Feltz, 2017]. Polycaprolactone (PCL), a biocompatible polymer, was used as the base material to make the fibers [Terrell, 2020; Chen, 2018]. Meanwhile, cut pieces of porcine liver, which have proven to be similar to the human liver for various biological applications [Ntonas, 2020; Lada, 2020], were decellularized to collect the native decellularized ECM (dECM), which was then ground, digested by pepsin and lyophilized. This dECM was added to the PCL solution before electrospinning so that the fibers would contain the native ECM components. Ideally, a larger ratio of the native dECM to PCL would be more biorelevant. However, it was observed that if the native dECM exceeded the amount of PCL (weight), the dECM started to precipitate and form layers while in the electrospinning solution. Therefore, the solution was made with 10% PCL (w/v) and 10% (w/v) dECM, which remained homogenous for electrospinning.

FIG. 2A is a Scanning Electron Microscopic (SEM) view of the composite (PCL+dECM) fibers, and FIG. 2B shows the fiber diameter distribution. As shown in FIGS. 1A-1D, fibers were deposited onto a polystyrene (PS) sheet with precut holes. After electrospinning, the sheet with fibers was laser-cut again around the outside of each hole to generate circular inserts. What remained was a PS ring with a layer of nanofibers on top, fused by the heat generated from the laser cutting. A total of 38 dECM inserts could be prepared in one electrospinning session, which enhanced efficiency and enabled experiments to be conducted on inserts from the same batch, reducing possible variations. FIG. 2C compares two inserts: PCL-only fibers (left) and PCL+dECM fibers (right). The inserts were incubated with Picrosirius red, a common dye in histology to stain collagen. After thorough rinsing, the dECM insert remained red, compared to the PCL-only fibers which were white. Because Picrosirius red is moderately fluorescent, the inserts were scanned with a plate reader and observed fluorescent signal heatmaps on the fibers with dECM (bottom panel of FIG. 2C). Further, fluorescent microscopic views of the composite (PCL+dECM) fibers stained with Picrosirius red, and staining by collagen I antibodies conjugated with CoraLite 488 of the composite fibers were obtained (not shown). Moreover, Fourier Transform Infrared (FTIR) spectroscopy was conducted over the fiber layers. As shown in FIG. 2D, plain PCL fibers showed clusters of peaks between 1000-1200 cm⁻¹ and 1400-1500 cm⁻¹, mainly corresponding to —C—O stretching and —C—H bending in PCL. However, with the inclusion of the dECM in the electrospinning solution, the resulting fibers showed a broad FTIR peak above 3000 cm⁻¹ (arrow), a typical signal of —OH groups. New peaks at 1540 cm⁻¹ and 1640 cm⁻¹ strongly indicate secondary amide (peptide bonds; inset of FIG. 2D). These FTIR peaks suggested the presence of proteins and thus further confirmed the successful inclusion of the native dECM in the fibers.

An insert holder was created to help seed cells onto the dECM insert and house them in a 12-well plate, as detailed in FIGS. 3A-3D. FIG. 3A demonstrates the assembly of the dECM insert into the 3D-printed insert holder device, with the bottom insert holder and a top hollow lid. After a circular insert was placed in the holder, a silicone O-ring was placed onto the insert before the hollow lid was screwed into the insert holder via the printed threads. FIGS. 3B and 3C show the CAD design views of the lid and holder, respectively, and FIG. 3D shows an assembled device. The entire assembly fits in a standard 12-well plate (FIG. 3E), demonstrating it can easily be integrated into the cell culture workflow of most laboratories. The 3D-printed parts and O-rings can be reused after rinsing and autoclaving to reduce plastic waste and cost. In the discussion below, hepatocytes cultured on the composite (PCL+dECM) nanofibrous scaffold are denoted as “on dECM,” and cells cultured 2D in a regular 12-well plate well are “on flat.”

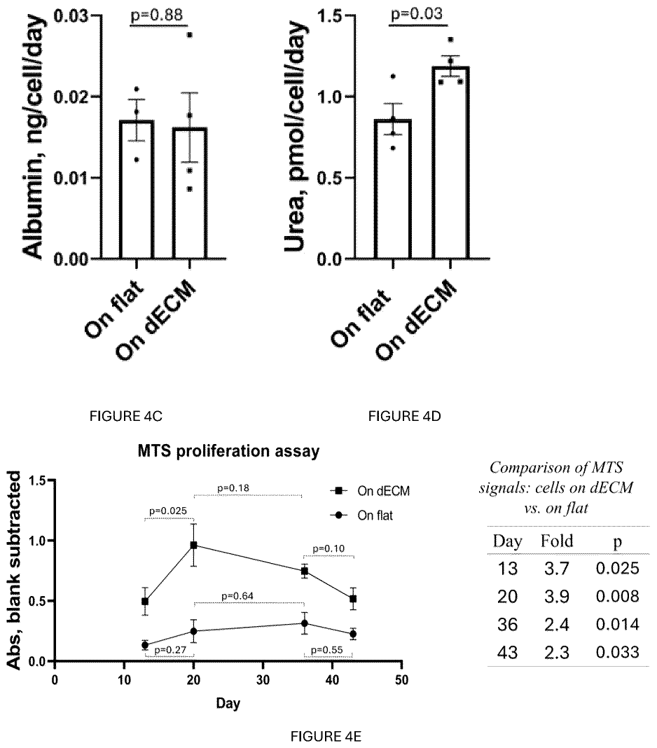

Fundamental characterization of the cells. HepaRG cells were evaluated after 13-14 days of differentiation on the fibrous dECM inserts. As shown in FIG. 4A, although the number of cells on dECM appeared to be less than on flat, the difference was not significant. There were over 200,000 cells for both conditions. FIG. 4B shows confocal microscopic views (top and sides) of hepatocytes in the scaffold. Clearly, the cells could infiltrate by ~50 μm into the fibrous layer to form a 3D culture. Hepatocytes were characterized by the ability to synthesize albumin and produce urea, two key fundamental functions of the cells. As suggested by FIGS. 4C and 4D, cells on both culture substrates showed albumin and urea production, indicating successful differentiation of HepaRG progenitors to hepatocytes. Interestingly, significant differences in albumin production from cells on flat versus on dECM were not observed, suggesting that some biosynthesis pathways of HepaRG hepatocytes might not be affected by the ECM. In contrast, cells on dECM produced significantly more urea than on flat, indicating that the metabolic activities of the hepatocytes might be enhanced by the ECM. Further experiments aligned with this observation, where the metabolic activity as measured by the MTS assay (FIG. 4E) was significantly higher from cells on dECM than on flat for up to 43 days. Between Day 13 and Day 20, cells on both substrates showed increased MTS metabolism (although not significant for cells on flat), and thus, cells within this time frame were used for the following studies.

Drug clearance investigations. To explore the scaffold's possible role in enhancing drug metabolism/clearance by the HepaRG hepatocytes, drugs were chosen that 1) need to be metabolized by the liver before excretion, with limited direct excretion, and 2) are well-studied and well-documented with abundant in vivo data available in the literature. Three drugs were chosen that are primarily metabolized by different enzymes in the hepatocyte: lidocaine (primarily metabolized by CYP3A4), clozapine (primarily metabolized by CYP1A2), and fluoxetine (primarily metabolized by CYP2D6).

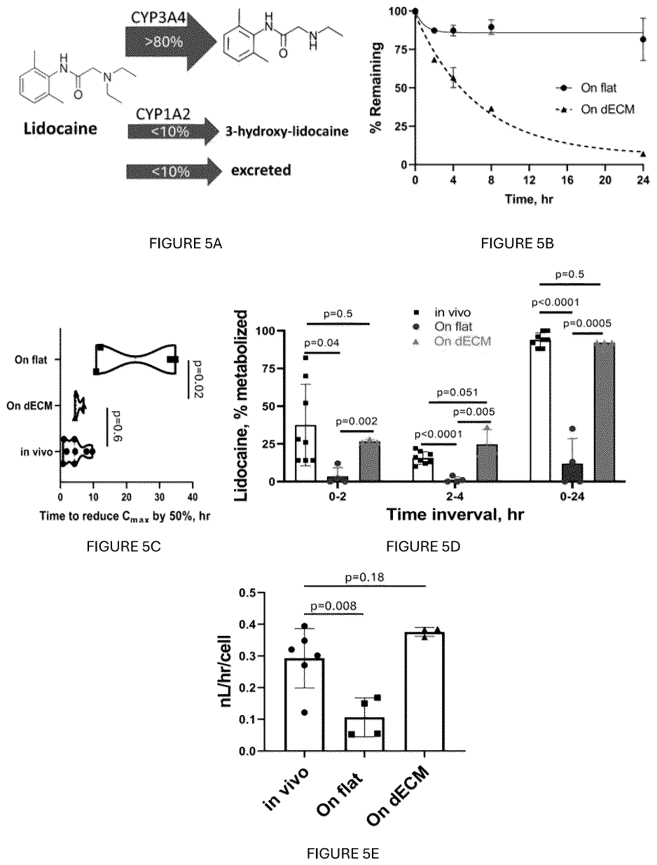

The first drug tested was lidocaine, a common anesthetic, which is mainly metabolized by CYP3A4 to monoethylglycinexylidide (MEGX), with less than 10% direct excretion [Torp, 2022](FIG. 5A). FIG. 5B depicts the concentration of lidocaine in the media of hepatocytes cultured on flat and on dECM as a function of time. The cells on dECM continued to clear the drug for 24 hours, while those on flat stopped after only 2 hours. By comparing the time needed to reduce the concentration to 50% of the peak level, the hepatocytes on dECM did not show a significant difference from in vivo, but cells on flat were significantly slower (FIG. 5C). A more detailed comparison at different time intervals (FIG. 5D) further validated that the hepatocytes on flat were significantly slower in lidocaine clearance than in vivo and cells on dECM. After 24 hours, hepatocytes on dECM cleared ~80% of lidocaine, comparable to in vivo results, but cells on flat could only clear ~14%. Intrinsic clearance was also calculated (FIG. 5E), with the in vivo clearance averaging 0.30 nL/hr/cell. The model used with HepaRG hepatocytes on dECM had 0.37 nL/hr/cell, and the conventional well plate model (on flat) cleared lidocaine at only 0.10 nL/hr/cell. Statistical analyses revealed no significant difference between HepaRG hepatocytes on dECM and in vivo, but the clearance by cells on flat was significantly lower.

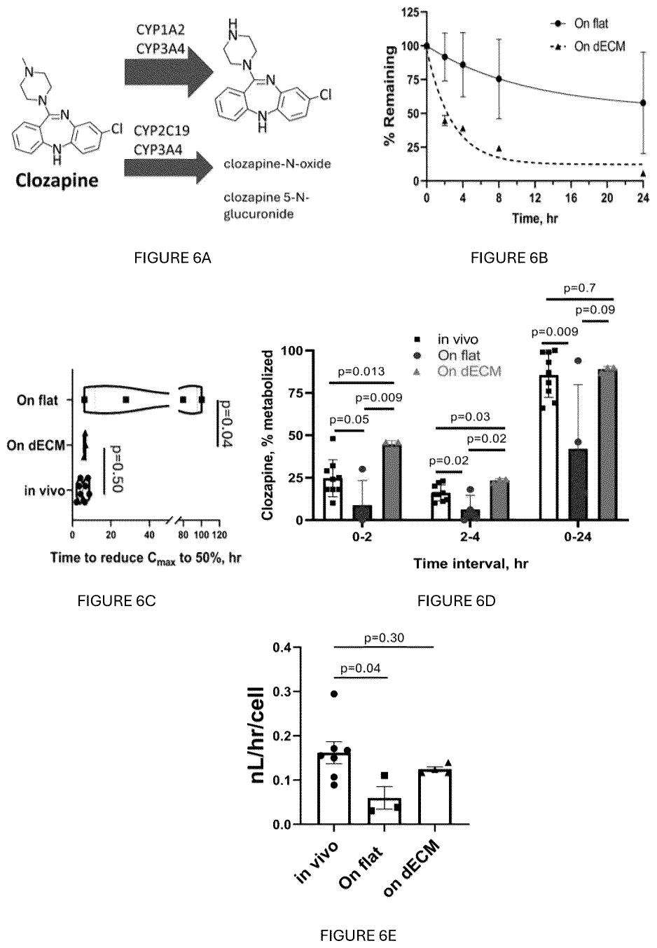

The next drug investigated was clozapine, an antipsychotic medication, which needs to be fully metabolized before excretion [Marinho, 2024](FIG. 6A). Clozapine is primarily metabolized by CYP1A2 and CYP3A4 to norclozapine. As with lidocaine, faster clearance of the drug was observed by the hepatocytes on dECM than on flat (FIG. 6B). The error bars for the flat controls were large because it was found that the cells started to die with the clozapine, but not the cells on dECM, even with the same drug concentrations. As shown in FIG. 6C, the time needed to reduce the highest concentration of clozapine to half was not significantly different between cells on dECM and in vivo data, while the time was statistically and dramatically longer for cells on flat. Comparisons with the more detailed time intervals (FIG. 6D) showed the same trends and alignments. Analyses of intrinsic clearance (FIG. 6E) also confirmed that the dECM model described herein was physiologically relevant, with the flat well-plate model significantly less capable.

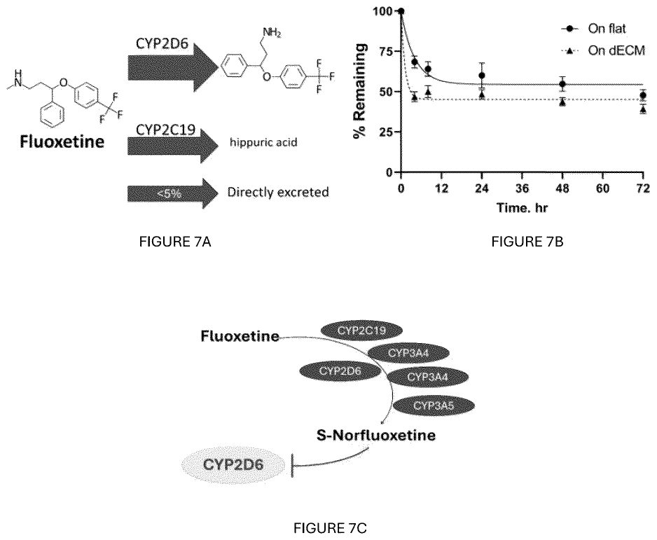

The third drug investigated was fluoxetine, a selective serotonin reuptake inhibitor commonly used to treat depression. As shown in FIG. 7A, over 95% of fluoxetine needs to be metabolized before excretion [Deodhar, 2021]. After measuring the fluoxetine in the media over 72 hours, the results shown in FIG. 7B were observed. Although hepatocytes cultured on dECM cleared more fluoxetine in the first four hours than cells on flat, the clearance stopped for both thereafter. The stopped clearance was likely caused by metabolites of fluoxetine, such as S-norfluoxetine, which can inhibit CYP2D6 [Id.], the key enzyme for fluoxetine metabolism (FIG. 7C). These data highlight the necessity of including permeable excretion models to couple with liver models in future studies for efficient removal of metabolites, as occurs in vivo, for certain drug clearance studies.

### CONCLUSION

It has been demonstrated that a composite nanofibrous scaffold composed of PCL and native porcine liver ECM components enabled enhanced metabolic activities of HepaRG hepatocytes compared to conventional 2D cultures. Combined with a modular 3D-printed holder device fitting in a standard 12-well plate, this hepatic model can easily be integrated into existing cell culture workflows. With this 3D cell culture model, well-documented drugs were tested with in vivo data and it was found that the 3D model significantly enhanced drug clearance versus the 2D well-plate model. Advantageously, the drug clearance times and rates on our model were comparable with published in vivo data without significant differences. This 3D model thus holds promises to facilitate hepatocyte studies during preclinical drug development, especially given that it reproduced the in vivo observations with a widely accessible cell source (HepaRG), rather than primary human hepatocytes or stem cells.

Although the experiments described herein were restricted to a single cell type, the modular holder can potentially support multiple stacked inserts, allowing co-culture of multiple cell types in standard well plates. For instance, a holder could house an insert seeded with HepaRG hepatocytes and a second contacting insert seeded with stellate cells—this co-culture cell model can mimic a real organ more closely to provide pharmacokinetic and biomedical insights. Also, the well plate setup makes it possible to test multiple drugs in different wells in one experiment.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

### CLAUSES

Clause 1: A method of producing a three-dimensional composite nanofibrous scaffold comprising native extracellular matrix (ECM) components, said method comprising:
electrospinning a polymer-containing solution onto a collector to produce a uniform, three-dimensional layer of nanofibers,
wherein the polymer-containing solution comprises polycaprolactone (PCL) and pepsinated native decellularized extracellular matrix (dECM) powder, and wherein the nanofibers comprise native ECM components, and wherein the nanofibers comprise native ECM components.

Clause 2: The method of clause 1, wherein the dECM is derived from native tissues.

Clause 3: The method of clauses 1 or 2, wherein the dECM is derived from native liver tissue.

Clause 4: The method of clause 3, wherein the native liver tissue is porcine liver tissue.

Clause 5: The method of any of clauses 2-4, wherein the pepsinated native dECM powder is obtained by a method comprising:
decellularizing the native tissues to obtain dECM;
lyophilizing and grinding the dECM to obtain powdered dECM;
digesting the powdered dECM with digestion solution to obtain a digested dECM; and
lyophilizing and grinding the digested dECM to obtain the pepsinated native dECM powder.

Clause 6: The method of clause 5, wherein the digestion solution comprises pepsin and at least one acid.

Clause 7: The method of any of clauses 1-6, wherein the polymer-containing solution further comprises 2,2,2-trifluoroethanol (TFE).

Clause 8: The method of any of clauses 1-7, wherein the polymer-containing solution further comprises TFE and at least one weak acid.

Clause 9: The method of any of clauses 1-8, wherein the ratio of PCL to dECM in the polymer-containing solution is about 1-5:1 (w/v), preferably about 1:1 (w/v).

Clause 10: The method of any of clauses 1-9, wherein the diameter of the nanofibers is in a range of about 100-500 nm.

Clause 11: The method of any of clauses 1-10, wherein the composite nanofibrous scaffold is devoid of hydrogel or hydrogel-based material.

Clause 12: The method of any of clauses 1-11, wherein the composite nanofibrous scaffold is devoid of one or more of hydrogel or a hydrogel-based material, alginate, polyvinyl alcohol (PVA) nanofibers, MATRIGEL® and/or GELTREX®, gelatin or a gelatin-based material, a polyethylene glycol (PEG)-based material, and any combination thereof.

Clause 13: The method of any of clauses 1-12, wherein the composite nanofibrous scaffold further comprises a polystyrene (PS) ring having a layer of nanofibers thereon.

Clause 14: A three-dimensional composite nanofibrous scaffold prepared according to the method of any of clauses 1-13.

Clause 15: A three-dimensional composite nanofibrous scaffold comprising nanofibers comprising polycaprolactone (PCL) and extracellular matrix (ECM) components of a native tissue.

Clause 16: The scaffold of clause 15, wherein the native tissue is native liver tissue.

Clause 17: The scaffold of clause 16, wherein the native liver tissue is porcine liver tissue.

Clause 18: The scaffold of any of clauses 14-17, wherein the diameter of the nanofibers is in a range of about 100-500 nm.

Clause 19: The scaffold of any of clauses 14-18, wherein the composite nanofibrous scaffold is devoid of hydrogel or hydrogel-based material.

Clause 20: The scaffold of any of clauses 14-18, wherein the composite nanofibrous scaffold is devoid of one or more of hydrogel or a hydrogel-based material, alginate, polyvinyl alcohol (PVA) nanofibers, MATRIGEL® and/or GELTREX®, gelatin or a gelatin-based material, a polyethylene glycol (PEG)-based material, and any combination thereof.

Clause 21: A three-dimensional tissue comprising the composite nanofibrous scaffold of any of clauses 14-20 and differentiated cells on and/or within the scaffold.

Clause 22: The three-dimensional tissue of clause 21, wherein the differentiated cells includes hepatocytes.

Clause 23: A method of making a three-dimensional tissue comprising a composite nanofibrous scaffold and cells, said method comprising:
electrospinning a polymer-containing solution onto a collector to produce a uniform, three-dimensional layer of nanofibers; and
culturing a cell line on and/or within the three-dimensional layer of nanofibers,
wherein the polymer-containing solution comprises polycaprolactone (PCL) and pepsinated native decellularized extracellular matrix (dECM) powder, and wherein the nanofibers comprise native ECM components, and
wherein the cells infiltrate into the three-dimensional layer of nanofibers.

Clause 24: The method of clause 23, wherein the dECM is derived from native tissues.

Clause 25: The method of clauses 23 or 24, wherein the dECM is derived from native liver tissues.

Clause 26: The method of clause 25, wherein the native liver tissue is porcine liver tissue.

Clause 27: The method of any of clauses 23-26, wherein the polymer-containing solution comprises 2,2,2-trifluoroethanol (TFE).

Clause 28: The method of any of clauses 23-27, wherein the polymer-containing solution comprises TFE and at least one weak acid.

Clause 29: The method of any of clauses 23-28, wherein the ratio of PCL to dECM in the polymer-containing solution is about 1-5:1 (w/v), preferably about 1:1 (w/v).

Clause 30: The method of any of clauses 23-29, wherein the diameter of the nanofibers is in a range of about 100-500 nm.

Clause 31: The method of any of clauses 23-30, wherein the composite nanofibrous scaffold is devoid of hydrogel or hydrogel-based material.

Clause 32: The method of any of clauses 23-31, wherein the composite nanofibrous scaffold is devoid of one or more of hydrogel or a hydrogel-based material, alginate, polyvinyl alcohol (PVA) nanofibers, MATRIGEL® and/or GELTREX®, gelatin or a gelatin-based material, a polyethylene glycol (PEG)-based material, and any combination thereof.

Clause 33: The method of any of clauses 23-32, wherein the composite nanofibrous scaffold further comprises a polystyrene (PS) ring having a layer of nanofibers thereon.

Clause 34: The method of any of clauses 23-33, wherein the cell line that is cultured is an immortalized human hepatic progenitor that is hepatocellular carcinoma-derived.

Clause 35: The method of any of clauses 23-33, wherein the cell line that is cultured is HepaRG.

Clause 36: The method of clauses 34 or 35, wherein the cells that infiltrate into the three-dimensional layer of nanofibers are hepatocytes.

Clause 37: A three-dimensional tissue comprising a composite nanofibrous scaffold and cells prepared according to the method of any of clauses 23-36.

Clause 38: A method of determining the extent of drug metabolism or clearance using the three-dimensional tissue of any of clauses 14-22 or 37 as an in vitro model.

### REFERENCES

Baiocchini, A.; Montaldo, C.; Conigliaro, A.; Grimaldi, A.; Correani, V.; Mura, F.; Ciccosanti, F.; Rotiroti, N.; Brenna, A.; Montalbano, M.; et al. Extracellular Matrix Molecular Remodeling in Human Liver Fibrosis Evolution. PLoS One 2016, 11 (3), e0151736. DOI: 10.1371/journal.pone.0151736
Beller, N. C.; Wang, Y.; Hummon, A. B. Evaluating the Pharmacokinetics and Pharmacodynamics of Chemotherapeutics within a Spatial SILAC-Labeled Spheroid Model System. Anal Chem 2023, 95 (30), 11263-11272. DOI: 10.1021/acs.analchem.3c00905
Chen, C.; Townsend, A. D.; Hayter, E. A.; Birk, H. M.; Sell, S. A.; Martin, R. S. Insert-based microfluidics for 3D cell culture with analysis. Analytical and Bioanalytical Chemistry 2018, 410 (12), 3025-3035. DOI: 10.1007/s00216-018-0985-y (acccessed 2023-06-17 17:19:01).
Deodhar, M.; Rihani, S. B. A.; Darakjian, L.; Turgeon, J.; Michaud, V. Assessing the Mechanism of Fluoxetine-Mediated CYP2D6 Inhibition. Pharmaceutics 2021, 13 (2). DOI: 10.3390/pharmaceutics13020148
Dityatev, A.; Schachner, M.; Sonderegger, P. The dual role of the extracellular matrix in synaptic plasticity and homeostasis. Nat Rev Neurosci 2010, 11 (11), 735-746. DOI: 10.1038/nrn2898
Dutta, R. C.; Dutta, A. K. Cell-interactive 3D-scaffold; advances and applications. Biotechnol Adv 2009, 27 (4), 334-339. DOI: 10.1016/j.biotechadv.2009.02.002
Feltz, K. P.; Growney Kalaf, E. A.; Chen, C.; Martin, R. S.; Sell, S. A. A review of electrospinning manipulation techniques to direct fiber deposition and maximize pore size. Electrospinning 2017, 2 (1), 46-61. DOI: 10.1515/esp-2017-0002 (acccessed 2023-06-17 17:17:48).
Gerardo-Nava, J. L.; Jansen, J.; Gunther, D.; Klasen, L.; Thiebes, A. L.; Niessing, B.; Bergerbit, C.; Meyer, A. A.; Linkhorst, J.; Barth, M.; et al. Transformative Materials to Create 3D Functional Human Tissue Models In Vitro in a Reproducible Manner. Adv Healthc Mater 2023, 12 (20), e2301030. DOI: 10.1002/adhm.202301030
Guillouzo, A.; Corlu, A.; Aninat, C.; Glaise, D.; Morel, F.; Guguen-Guillouzo, C. The human hepatoma HepaRG cells: a highly differentiated model for studies of liver metabolism and toxicity of xenobiotics. Chem Biol Interact 2007, 168 (1), 66-73. DOI: 10.1016/j.cbi.2006.12.003
Gupta, S.; Sharma, A.; Rajakannu, M.; Bisevac, J.; Rela, M.; Verma, R. S. Small Molecule-Mediated Stage-Specific Reprogramming of MSCs to Hepatocyte-Like Cells and Hepatic Tissue for Liver Injury Treatment. Stem Cell Rev Rep 2024, 20 (8), 2215-2235. DOI: 10.1007/s12015-024-10771-x
Hedaya, M. A. Basic pharmacokinetics; Routledge, 2023
Huang, T.; Jones, C. G.; Chung, J. H.; Chen, C. Microfibrous Extracellular Matrix Changes the Liver Hepatocyte Energy Metabolism via Integrins. ACS Biomater Sci Eng 2020, 6 (10), 5849-5856. DOI: 10.1021/acsbiomaterials.0c01311
Huang, T.; Terrell, J. A.; Chung, J. H.; Chen, C. Electrospun Microfibers Modulate Intracellular Amino Acids in Liver Cells via Integrin β1. Bioengineering 2021, 8 (7), 88. DOI: 10.3390/bioengineering8070088
Hussey, G. S.; Dziki, J. L.; Badylak, S. F. Extracellular matrix-based materials for regenerative medicine. Nature Reviews Materials 2018, 3 (7), 159-173. DOI: 10.1038/s41578-018-0023-x
Jackson, J. P.; Li, L.; Chamberlain, E. D.; Wang, H.; Ferguson, S. S. Contextualizing Hepatocyte Functionality of Cryopreserved HepaRG Cell Cultures. Drug Metab Dispos 2016, 44 (9), 1463-1479. DOI: 10.1124/dmd.116.069831
Jones, C. G.; Huang, T.; Chung, J. H.; Chen, C. 3D-Printed, Modular, and Parallelized Microfluidic System with Customizable Scaffold Integration to Investigate the Roles of Basement Membrane Topography on Endothelial Cells. ACS Biomaterials Science & Engineering 2021, 7 (4), 1600-1607. DOI: 10.1021/acsbiomaterials.0c01752
Kanebratt, K. P.; Andersson, T. B. Evaluation of HepaRG cells as an in vitro model for human drug metabolism studies. Drug Metab Dispos 2008, 36 (7), 1444-1452. DOI: 10.1124/dmd.107.020016
Karamanos, N. K.; Theocharis, A. D.; Piperigkou, Z.; Manou, D.; Passi, A.; Skandalis, S. S.; Vynios, D. H.; Orian-Rousseau, V.; Ricard-Blum, S.; Schmelzer, C. E. H.; et al. A guide to the composition and functions of the extracellular matrix. FEBS J 2021, 288 (24), 6850-6912. DOI: 10.1111/febs.15776
Kolesky, D. B.; Homan, K. A.; Skylar-Scott, M. A.; Lewis, J. A. Three-dimensional bioprinting of thick vascularized tissues. Proceedings of the National Academy of Sciences 2016, 113 (12), 3179-3184. DOI: 10.1073/pnas.1521342113 (acccessed 2022-03-06 00:16:49).
Kvist, A. J.; Kanebratt, K. P.; Walentinsson, A.; Palmgren, H.; O'Hara, M.; Bjorkbom, A.; Andersson, L. C.; Ahlqvist, M.; Andersson, T. B. Critical differences in drug metabolic properties of human hepatic cellular models, including primary human hepatocytes, stem cell derived hepatocytes, and hepatoma cell lines. Biochem Pharmacol 2018, 155, 124-140. DOI: 10.1016/j.bcp.2018.06.026
Lada, E.; Anna, M.; Patrik, M.; Zbynek, T.; Miroslav, J.; Hynek, M.; Richard, P.; Sarah, L.; Vaclav, L. Porcine Liver Anatomy Applied to Biomedicine. J Surg Res 2020, 250, 70-79. DOI: 10.1016/j.jss.2019.12.038
Leung, C. M.; de Haan, P.; Ronaldson-Bouchard, K.; Kim, G.-A.; Ko, J.; Rho, H. S.; Chen, Z.; Habibovic, P.; Jeon, N. L.; Takayama, S.; et al. A guide to the organ-on-a-chip. Nature Reviews Methods Primers 2022, 2 (1). DOI: 10.1038/s43586-022-00118-6
Lin, J.; Sahakian, D. C.; de Morais, S. M.; Xu, J. J.; Polzer, R. J.; Winter, S. M. The role of absorption, distribution, metabolism, excretion and toxicity in drug discovery. Curr Top Med Chem 2003, 3 (10), 1125-1154. DOI: 10.2174/1568026033452096
Low, L. A.; Mummery, C.; Berridge, B. R.; Austin, C. P.; Tagle, D. A. Organs-on-chips: into the next decade. Nat Rev Drug Discov 2021, 20 (5), 345-361. DOI: 10.1038/s41573-020-0079-3
Mazza, G.; Rombouts, K.; Rennie Hall, A.; Urbani, L.; Vinh Luong, T.; Al-Akkad, W.; Longato, L.; Brown, D.; Maghsoudlou, P.; Dhillon, A. P.; et al. Decellularized human liver as a natural 3D-scaffold for liver bioengineering and transplantation. Sci Rep 2015, 5, 13079. DOI: 10.1038/srep13079
Marinho, E. Clozapine: A special case of an atypical antipsychotic. European Journal of Medicinal Chemistry Reports 2024, 10. DOI: 10.1016/j.ejmcr.2024.100140
Molina, D. K.; DiMaio, V. J. M. Normal Organ Weights in Men: Part II—The Brain, Lungs, Liver, Spleen, and Kidneys. The American Journal of Forensic Medicine and Pathology 2012, 33 (4), 368-372. DOI: 10.1097/PAF.0b013e31823d29ad
Ning, M.; Duarte, J. D.; Rubin, L. H.; Jeong, H. CYP2D6 Protein Level Is the Major Contributor to Interindividual Variability in CYP2D6-Mediated Drug Metabolism in Healthy Human Liver Tissue. Clin Pharmacol Ther 2018, 104 (5), 974-982. DOI: 10.1002/cpt.1032
Ning, M.; Duarte, J. D.; Stevison, F.; Isoherranen, N.; Rubin, L. H.; Jeong, H. Determinants of Cytochrome P450 2D6 mRNA Levels in Healthy Human Liver Tissue. Clin Transl Sci 2019, 12 (4), 416-423. DOI: 10.1111/cts.12632
Ntonas, A.; Katsourakis, A.; Galanis, N.; Filo, E.; Noussios, G. Comparative Anatomical Study Between the Human and Swine Liver and Its Importance in Xenotransplantation. Cureus 2020, 12 (7), e9411. DOI: 10.7759/cureus.9411
Peaston, A. E.; Whitelaw, E. Epigenetics and phenotypic variation in mammals. Mamm Genome 2006, 17 (5), 365-374. DOI: 10.1007/s00335-005-0180-2
Sackett, S. D.; Tremmel, D. M.; Ma, F.; Feeney, A. K.; Maguire, R. M.; Brown, M. E.; Zhou, Y.; Li, X.; O'Brien, C.; Li, L.; et al. Extracellular matrix scaffold and hydrogel derived from decellularized and delipidized human pancreas. Sci Rep 2018, 8 (1), 10452. DOI: 10.1038/s41598-018-28857-1
Saraswathibhatla, A.; Indana, D.; Chaudhuri, O. Cell-extracellular matrix mechanotransduction in 3D. Nat Rev Mol Cell Biol 2023, 24 (7), 495-516. DOI: 10.1038/s41580-023-00583-1
Serras, A. S.; Rodrigues, J. S.; Cipriano, M.; Rodrigues, A. V.; Oliveira, N. G.; Miranda, J. P. A Critical Perspective on 3D Liver Models for Drug Metabolism and Toxicology Studies. Front Cell Dev Biol 2021, 9, 626805. DOI: 10.3389/fcell.2021.626805
Stanley, L. A.; Wolf, C. R. Through a glass, darkly? HepaRG and HepG2 cells as models of human phase I drug metabolism. Drug Metab Rev 2022, 54 (1), 46-62. DOI: 10.1080/03602532.2022.2039688
Terrell, J. A.; Jones, C. G.; Kabandana, G. K. M.; Chen, C. From cells-on-a-chip to organs-on-a-chip: scaffolding materials for 3D cell culture in microfluidics. Journal of Materials Chemistry B 2020, 8 (31), 6667-6685. DOI: 10.1039/DOTB00718H (acccessed 2023-06-17 17:17:05).
Torp K D, M. E., Simon LV. Lidocaine Toxicity. StatPearls Publishing, 2022.
Wilson, Z. E.; Rostami-Hodjegan, A.; Burn, J. L.; Tooley, A.; Boyle, J.; Ellis, S. W.; Tucker, G. T. Inter-individual variability in levels of human microsomal protein and hepatocellularity per gram of liver. Br J Clin Pharmacol 2003, 56 (4), 433-440. DOI: 10.1046/j.1365-2125.2003.01881.x
Xue, J.; Wu, T.; Dai, Y.; Xia, Y. Electrospinning and Electrospun Nanofibers: Methods, Materials, and Applications. Chem Rev 2019, 119 (8), 5298-5415. DOI: 10.1021/acs.chemrev.8b00593

What is claimed is:

1. A method of producing a three-dimensional composite nanofibrous scaffold comprising native extracellular matrix (ECM) components, said method comprising:
electrospinning a polymer-containing solution onto a collector to produce a uniform, three-dimensional layer of nanofibers,
wherein the polymer-containing solution comprises polycaprolactone (PCL) and pepsinated native decellularized extracellular matrix (dECM) powder, and wherein the nanofibers comprise native ECM components, and wherein the nanofibers comprise native ECM components.

2. The method of claim 1, wherein the dECM is derived from native tissues.

3. The method of claim 1, wherein the dECM is derived from native liver tissue.

4. The method of claim 3, wherein the native liver tissue is porcine liver tissue.

5. The method of claim 2, wherein the pepsinated native dECM powder is obtained by a method comprising:
decellularizing the native tissues to obtain dECM;
lyophilizing and grinding the dECM to obtain powdered dECM;
digesting the powdered dECM with digestion solution to obtain a digested dECM; and
lyophilizing and grinding the digested dECM to obtain the pepsinated native dECM powder.

6. The method of claim 5, wherein the digestion solution comprises pepsin and at least one acid.

7. The method of claim 1, wherein the polymer-containing solution further comprises 2,2,2-trifluoroethanol (TFE).

8. The method of claim 1, wherein the polymer-containing solution further comprises TFE and at least one weak acid.

9. The method of claim 1, wherein the ratio of PCL to dECM in the polymer-containing solution is about 1-5:1 (w/v).

10. The method of claim 1, wherein the diameter of the nanofibers is in a range of about 100-500 nm.

11. The method of claim 1, wherein the composite nanofibrous scaffold is devoid of one or more of hydrogel or a hydrogel-based material, alginate, polyvinyl alcohol (PVA) nanofibers, MATRIGEL® and/or GELTREX®, gelatin or a gelatin-based material, a polyethylene glycol (PEG)-based material, and any combination thereof.

12. The method of claim 1, wherein the composite nanofibrous scaffold further comprises a polystyrene (PS) ring having a layer of nanofibers thereon.

13. A three-dimensional composite nanofibrous scaffold comprising nanofibers comprising polycaprolactone (PCL) and extracellular matrix (ECM) components of a native tissue.

14. The scaffold of claim 13, wherein the diameter of the nanofibers is in a range of about 100-500 nm.

15. The scaffold of claim 13, wherein the composite nanofibrous scaffold is devoid of one or more of hydrogel or a hydrogel-based material, alginate, polyvinyl alcohol (PVA) nanofibers, MATRIGEL® and/or GELTREX®, gelatin or a gelatin-based material, a polyethylene glycol (PEG)-based material, and any combination thereof.

16. A method of making a three-dimensional tissue comprising a composite nanofibrous scaffold and cells, said method comprising:
culturing a cell line on and/or within the three-dimensional layer of nanofibers of claim 1, wherein the cells infiltrate into the three-dimensional layer of nanofibers.

17. The method of claim 16, wherein the composite nanofibrous scaffold is devoid of one or more of hydrogel or a hydrogel-based material, alginate, polyvinyl alcohol (PVA) nanofibers, MATRIGEL® and/or GELTREX®, gelatin or a gelatin-based material, a polyethylene glycol (PEG)-based material, and any combination thereof.

18. The method of claim 17, wherein the cell line that is cultured is an immortalized human hepatic progenitor that is hepatocellular carcinoma-derived.

19. The method of claim 17, wherein the cells that infiltrate into the three-dimensional layer of nanofibers are hepatocytes.

20. A three-dimensional tissue, said three-dimensional tissue comprising cells and a composite nanofibrous scaffold comprising nanofibers comprising polycaprolactone (PCL) and extracellular matrix (ECM) components of a native tissue, wherein the cells are present on and/or within the composite nanofibrous scaffold.
